(19) United States
(12) Patent Application Publication
(10) Pub. No.: US 2026/0090741 A1
(43) Pub. Date: Apr. 2, 2026

(54) LIVING BODY INFORMATION MEASURING APPARATUS

(71) Applicant: SEIKO EPSON CORPORATION, Tokyo (JP)

(72) Inventor: Hiroki KOBAYASHI, HARA-MURA (JP)

(73) Assignee: SEIKO EPSON CORPORATION, Tokyo (JP)

(21) Appl. No.: 19/337,936

(22) Filed: Sep. 24, 2025

(30) Foreign Application Priority Data
Sep. 27, 2024 (JP) 2024-168292

(51) Int. Cl. A61B 5/1455

(52) U.S. Cl. CPC A61B 5/14552

(57) ABSTRACT

A living body information measuring apparatus includes an enclosure having an enclosure surface that comes into contact with an object under inspection in a measurement state; a light measurement portion disposed in the enclosure and configured to radiate measurement light to the object under inspection and receive reflected light; a light transmissive member configured to transmit the measurement light and the reflected light, and including multiple protrusions configured to press the object under inspection in the measurement state, the multiple protrusions located at the contact surface; and a support portion disposed between the light transmissive member and the enclosure, and configured to come into contact with the light transmissive member to support the light transmissive member when a force greater than or equal to a predetermined threshold is applied by the object under inspection to the light transmissive member in the measurement state.

The present application is based on, and claims priority from JP Application Serial Number 2024-168292, filed September 27, 2024, the disclosure of which is hereby incorporated by reference herein in its entirety.

### BACKGROUND

### Technical Field

The present disclosure relates to a living body information measuring apparatus.

### Related Art

A living body information measuring apparatus of related art that measures living body information such as the pulse rate and the oxygen saturation in the arterial blood has been used. JP-A-2014-180289 discloses a living body information measuring apparatus that is attached to a wrist of an object under inspection and functions as what is called a wristwatch-shaped pulsimeter. The apparatus includes a light transmissive member facing the wrist, and a light measurement portion including a light emitter and a light receiver provided below the light transmissive member. To realize stable measurement, it is desired to suppress a change in the relative positional relationship between the living body information measuring apparatus and the object under inspection resulting from a motion of the object under inspection (hereinafter also referred to as "body motion") or the like. In the living body information measuring apparatus disclosed in JP-A-2014-180289, when the living body information measuring apparatus is attached to the wrist, the position where the band of the apparatus is attached is adjusted to press the apparatus against the object under inspection with a predetermined force. The influence of the body motion can therefore be readily suppressed.

JP-A-2014-180289 is an example of the related art.

In recent years, what is called a "patch-shaped" living body information measuring apparatus used in close contact with an arm or any other site of an object under inspection with the aid of a dressing member has been studied as a living body information measuring apparatus different from a wristwatch-shaped apparatus. Since such a patch-shaped living body information measuring apparatus is pressed against the object under inspection with the adhesive force produced by the dressing member, it is not easy to adjust the pressing force to a predetermined force. The light transmissive member therefore receives a reaction force greater than an intended force, so that the distance between the object under inspection and the light measurement portion is shortened, resulting in a problem of a decrease in measurement accuracy.

### SUMMARY

According to an aspect of the present disclosure, there is provided a living body information measuring apparatus used in close contact with an object under inspection with the aid of a dressing member. The living body information measuring apparatus includes: an enclosure having an enclosure surface that comes into contact with the object under inspection in a measurement state in which living body information on the object under inspection is measured; a light measurement portion disposed in the enclosure and configured to radiate measurement light to the object under inspection and receive reflected light; a light transmissive member configured to transmit the measurement light and the reflected light, and including multiple protrusions configured to press the object under inspection in the measurement state, the multiple protrusions located at the contact surface; and a support portion disposed between the light transmissive member and the enclosure, and configured to come into contact with the light transmissive member to support the light transmissive member when a force greater than or equal to a predetermined threshold is applied by the object under inspection to the light transmissive member in the measurement state.

### BRIEF DESCRIPTION OF THE DRAWINGS

FIG. 1 is a first perspective view showing an exterior configuration of a living body information measuring apparatus as an embodiment of the present disclosure.

FIG. 2 is a second perspective view showing the exterior configuration of the living body information measuring apparatus.

FIG. 3 illustrates a method for bringing the living body information measuring apparatus into close contact with an object under inspection.

FIG. 4 is an exploded perspective view showing a detailed configuration of the living body information measuring apparatus.

FIG. 5 is a cross-sectional view showing the detailed configuration of the living body information measuring apparatus.

FIG. 6 is a plan view showing an exterior shape of a first enclosure portion.

FIG. 7 is a partial cutaway view showing the configuration of the first enclosure portion.

FIG. 8 is a plan view showing a detailed configuration of a sensor substrate.

FIG. 9 is a partial cross-sectional view showing a detailed configuration of the living body information measuring apparatus.

FIG. 10 illustrates the positional relationship of four protrusions and a recess with a first measurement portion and a second measurement portion.

FIG. 11 illustrates the positional relationship between a beam portion in a second embodiment and each of the measurement portions.

### DESCRIPTION OF EMBODIMENTS

### A. First embodiment:

### A1. Schematic configuration of living body information measuring apparatus 10:

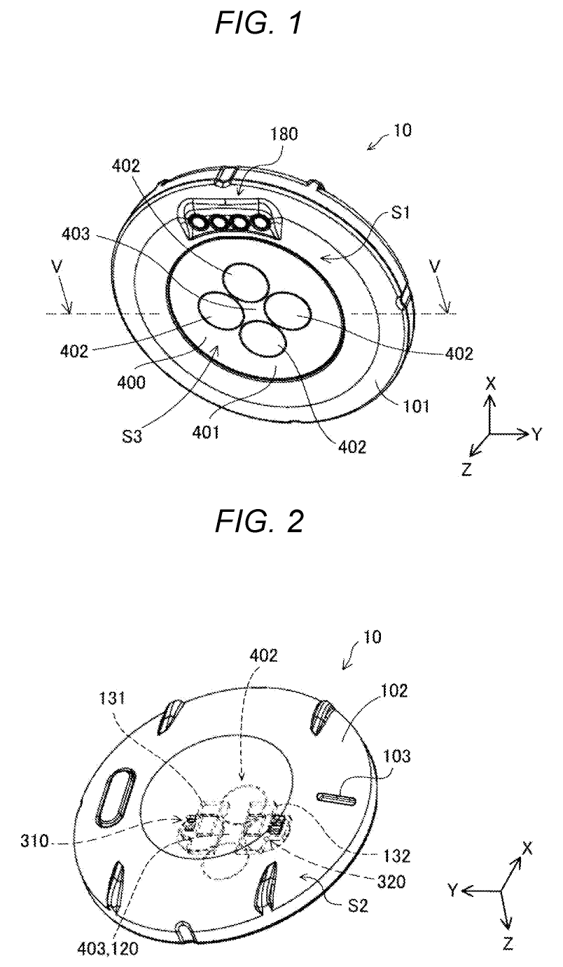

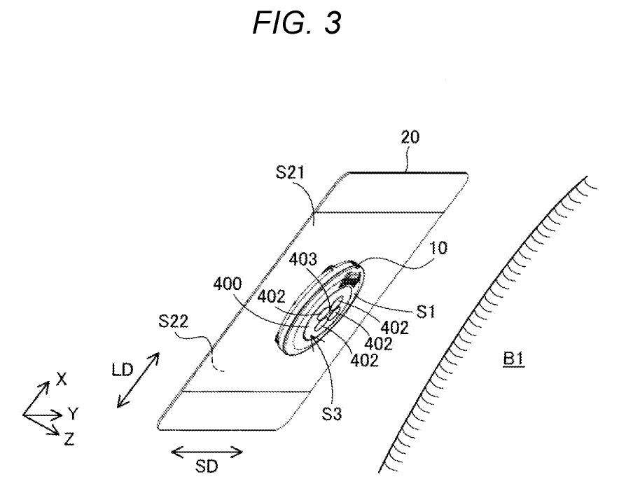

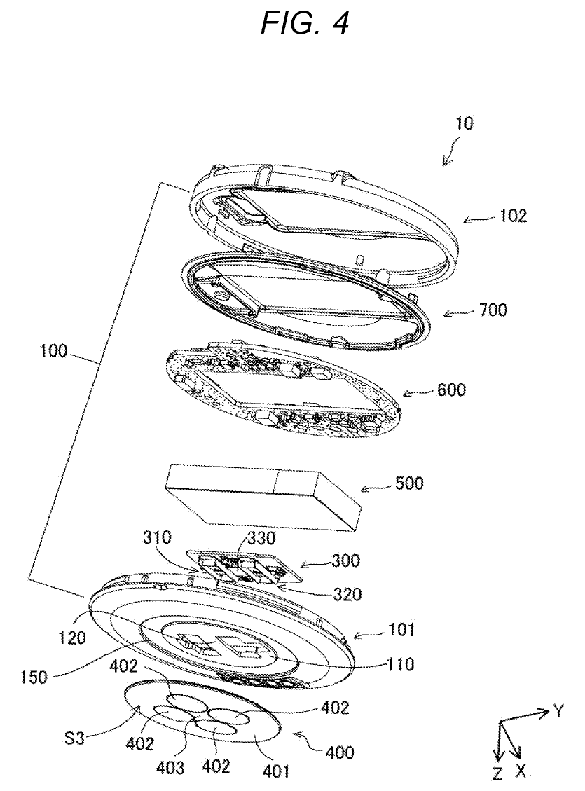

FIG. 1 is a first perspective view showing an exterior configuration of a living body information measuring apparatus 10 as an embodiment of the present disclosure. FIG. 2 is a second perspective view showing the exterior configuration of the living body information measuring apparatus 10. FIG. 3 illustrates a method for bringing the living body information measuring apparatus 10 into close contact with an object under inspection. Note that FIGS. 1 to 3 show an X-axis, a Y-axis, and a Z-axis orthogonal to each other. The same X-axis, Y-axis, and Z-axis are drawn in FIG. 4 and subsequent drawings. An "X-axis direction" is hereinafter a collective name of a +X direction and a −X direction. Similarly, a "Y-axis direction" is a collective name of a +Y direction and a −Y direction, and a "Z-axis direction" is a collective name of a +Z direction and a −Z direction.

The living body information measuring apparatus 10 is an apparatus that measures living body information on an object under inspection. In the present embodiment, the "living body information" means the pulse and the oxygen saturation (SpO₂) in the arterial blood. Note that the "living body information" is not limited to the pulse and the oxygen saturation in the arterial blood, and may be any other living body information on the object under inspection, such as the blood pressure, the total amount of in-blood hemoglobin, or the carbon monoxide concentration in the arterial blood. The living body information measuring apparatus 10 is used in close contact with an object under inspection B1 with the aid of a dressing member 20, as shown in FIG. 3. The object under inspection B1 corresponds, for example, to a human arm.

The living body information measuring apparatus 10 has a flat columnar (button-shaped) exterior, as shown in FIGS. 1 to 3. The thickness direction of the living body information measuring apparatus 10 is parallel to the Z-axis direction. An end surface of the living body information measuring apparatus 10 that is the end surface facing the positive end in the Z direction faces the object under inspection in a state in which living body information on the object under inspection is measured (hereinafter referred to as "measurement state"), as shown in FIGS. 1 and 3. The end surface has a contact surface S3 of a light transmissive member 400, which will be described later, and an annular enclosure surface S1, which surrounds the end surface of the light transmissive member 400. The enclosure surface S1 is a part of a first enclosure portion 101, which will be described later.

An end surface S2 of the living body information measuring apparatus 10 shown in FIG. 2, which is the end surface facing the negative end in the Z direction, is covered with the dressing member 20 in the measurement state. The end surface S2 is a part of a second enclosure portion 102, which will be described later. Note that FIG. 2 shows the following elements indicated by broken lines for convenience of the description: four protrusions 402 and a recess 403 of the light transmissive member 400 disposed on the side facing the positive end in the Z direction in the living body information measuring apparatus 10; two openings (first opening 131 and second opening 132) and a beam portion 120 formed in the first enclosure portion 101; and a first measurement portion 310 and a second measurement portion 320, which will be described later. The dressing member 20 is a thin-film-shaped member and is made, for example, of polyurethane. An adhesive having a predetermined adhesive force is applied to an attachment surface S21 of the dressing member 20. The living body information measuring apparatus 10 is attached to the attachment surface S21 with the second enclosure portion 102 facing the attachment surface S21. In this state, the attachment surface S21 is attached to the surface of the object under inspection B1. A surface S22 opposite the attachment surface S21 is a surface exposed to the outside in the measurement state.

A direction indicator 103 is provided at the end surface S2. In the present embodiment, the direction indicator 103 is configured as a groove extending in the Y-axis direction. The direction indicator 103 indicates the mounting direction of the living body information measuring apparatus 10. Specifically, the direction indicator 103 is used to indicate that the living body information measuring apparatus 10 should be so attached to the object under inspection B1 that the lengthwise direction of the direction indicator 103, that is, the Y-axis direction in which the direction indicator 103 extends is parallel to the widthwise direction of the arm, which is the object under inspection B1. In the present embodiment, the dressing member 20 has a rectangular shape in a plan view, as shown in FIG. 3. For example, the living body information measuring apparatus 10 is so attached to the dressing member 20 that the lengthwise direction of the direction indicator 103 is parallel to a widthwise direction SD of the dressing member 20, and with this state maintained, the dressing member 20 and the living body information measuring apparatus 10 are so attached to the object under inspection B1 that a lengthwise direction LD of the dressing member 20 coincides with the lengthwise direction of the object under inspection B1. The living body information measuring apparatus 10 can thus be attached in an intended posture to the object under inspection B1.

### A2. Detailed configuration of living body information measuring apparatus 10:

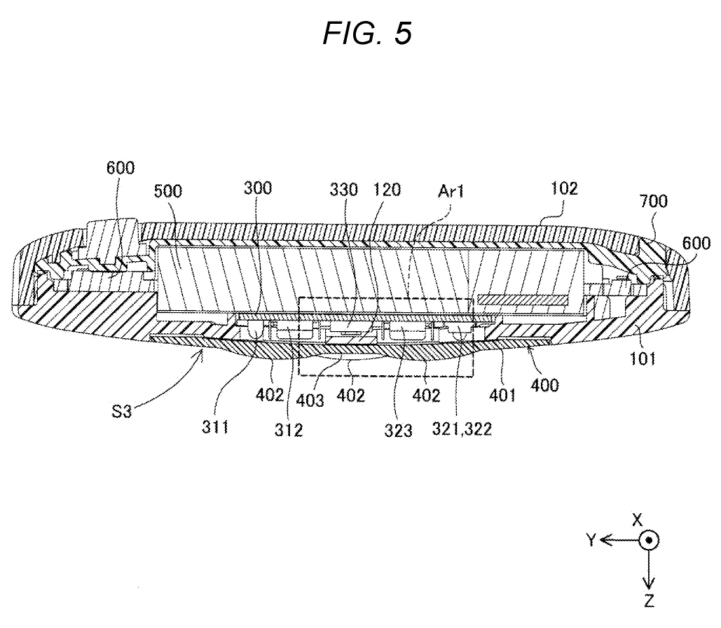

FIG. 4 is an exploded perspective view showing a detailed configuration of the living body information measuring apparatus 10. FIG. 5 is a cross-sectional view showing the detailed configuration of the living body information measuring apparatus 10. FIG. 5 shows the cross section of the living body information measuring apparatus 10 taken along the line V-V shown in FIG. 1.

The living body information measuring apparatus 10 includes an enclosure 100, a sensor substrate 300, the light transmissive member 400, a battery 500, a primary substrate 600, and a waterproof cover 700.

### ENCLOSURE 100

The enclosure 100 includes the first enclosure portion 101 and the second enclosure portion 102. The first enclosure portion 101 and the second enclosure portion 102 face each other in the Z-axis direction and are fitted to each other to form the enclosure 100. The first enclosure portion 101 may also be referred to as an upper cover, and the second enclosure portion 102 may also be referred to as a lower cover. The first enclosure portion 101 and the second enclosure portion 102 are made, for example, of polycarbonate.

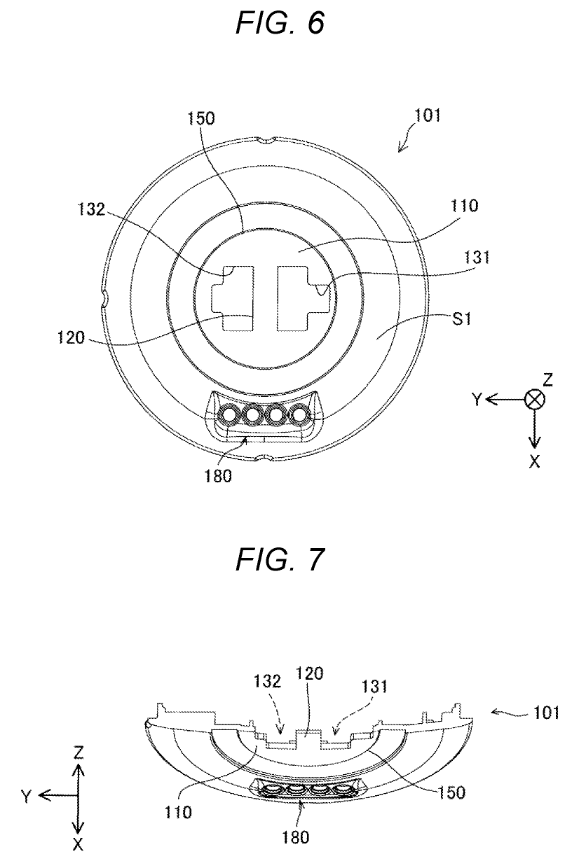

FIG. 6 is a plan view showing an exterior shape of the first enclosure portion 101. FIG. 6 is a plan view of the first enclosure portion 101 viewed in the +Z direction. FIG. 7 is a partial cutaway view showing the configuration of the first enclosure portion 101. FIG. 7 is a cutaway view of the first enclosure portion 101 cut along a plane parallel to the Y-axis direction and passing through the center position of the first enclosure portion 101 in the X-axis direction, and shows the portion on the side facing the positive end in the X direction.

The first enclosure portion 101 has a circular exterior shape when viewed in the Z-axis direction, as shown in FIG. 6. The enclosure surface S1 comes into contact with the object under inspection B1 in the measurement state. A terminal housing 180 having four openings arranged in the Y-axis direction is formed at an end portion of the first enclosure portion 101 that is an end portion facing the positive end in the X direction. The terminal housing 180 houses a terminal group that is not shown but allows the living body information measuring apparatus 10 to be electrically coupled to an external apparatus. For example, the terminal housing 180 houses a terminal to be coupled to a cable that allows the external apparatus to read living body information stored in a memory provided at the primary substrate 600.

A support portion 150 is provided at the center of the first enclosure portion 101 when viewed in the +Z direction. When a force greater than or equal to a predetermined threshold is applied by the object under inspection B1 to the light transmissive member 400, the support portion 150 comes into contact with the light transmissive member 400 and supports the light transmissive member 400. How the support portion 150 supports the light transmissive member 400 will be described later in detail. The support portion 150 is provided in the first enclosure portion 101 at a position corresponding to the light transmissive member 400 in the Z-axis direction. The support portion 150 includes a base portion 110 and the beam portion 120. The first opening 131 and the second opening 132 are formed in the base portion 110. The first opening 131 and the second opening 132 are disposed separate from each other in the Y-axis direction. The beam portion 120 is provided as a section between the first opening 131 and the second opening 132. In the present embodiment, the beam portion 120 has a plate-like exterior shape extending in the X-axis direction. The base portion 110 is continuous with the opposite ends of the beam portion 120 in the X-axis direction and supports the beam portion 120.

### SENSOR SUBSTRATE 300

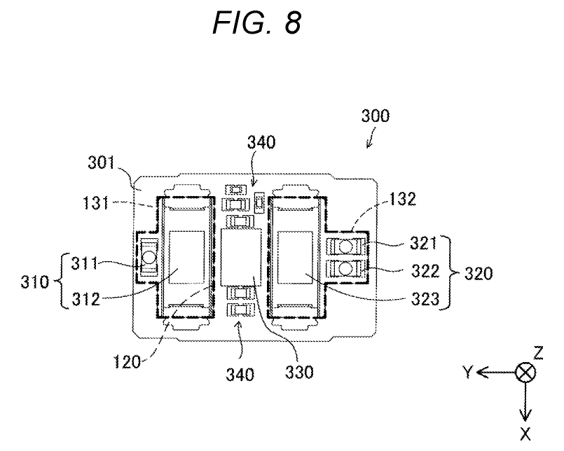

FIG. 8 is a plan view showing a detailed configuration of the sensor substrate 300. FIG. 8 is a plan view showing the sensor substrate 300 viewed in the +Z direction. Note in FIG. 8 that the two openings 131 and 132 described above are indicated by broken lines for convenience of the description. The sensor substrate 300 includes a substrate 301, and the following elements provided on the substrate 301: the first measurement portion 310; the second measurement portion 320; an analog front end (AFE) IC 330; and a semiconductor memory group 340. Note that the first measurement portion 310 and the second measurement portion 320 are also collectively referred to as a "light measurement portion".

The first measurement portion 310 measures the pulse. The first measurement portion 310 includes a first light radiator 311 and a first light receiver 312. In the present embodiment, the first light radiator 311 includes an LED that radiates green measurement light. The first light receiver 312 includes a photodiode that receives reflected light based on the green measurement light. The volume of a blood vessel changes in synchronization with the pulse, so that the amount of absorbed green measurement light changes. The intensity of the reflected light received by the first light receiver 312 therefore changes in synchronization with the pulse, so that the pulse can be measured by measuring the intensity of the reflected light.

The second measurement portion 320 measures the oxygen saturation (SpO₂) in the arterial blood. The second measurement portion 320 includes a second light radiator 321, a third light radiator 322, and a second light receiver 323. The second light radiator 321 includes an LED that radiates red measurement light. The third light radiator 322 includes an LED that radiates infrared light. The second light radiator 321 and the third light radiator 322 can be collectively referred to as a "second light radiator" to be distinguished from the first light radiator 311. The second light receiver 323 receives reflected light based on the red light, and reflected light based on the infrared light. The oxygen saturation (SpO₂) in the arterial blood can be measured based on the intensity ratio between the red reflected light and the infrared reflected light by making use of the fact that hemoglobin combined with oxygen absorbs a smaller amount of the red light than the amount of the red light absorbed by hemoglobin uncombined with oxygen.

The analog front end IC 330 has the function of performing A/D conversion on the intensity (analog value) of the light received by each of the first light receiver 312 and the second light receiver 323 and outputting the intensity in the form of a digital value, the function of calculating the ratio between the intensity of the reflected red light and the intensity of the reflected infrared light described above, and other functions. The semiconductor memory group 340 stores results of the measurement made by the first measurement portion 310 and the second measurement portion 320, that is, a result of output from the analog front end IC 330.

The analog front end IC 330 and the semiconductor memory group 340 are disposed at a central portion of the sensor substrate 300 in the Y-axis direction, as shown in FIG. 8. The first measurement portion 310 is disposed at a position shifted in the +Y direction from the analog front end IC 330 and the semiconductor memory group 340, and the second measurement portion 320 is disposed at a position shifted in the −Y direction therefrom. Note that shielding walls extending in the X-axis direction are provided on opposite sides of the first light receiver 312 in the Y direction, as shown in FIGS. 4, 5, and 8. Similarly, shielding walls extending in the X-axis direction are provided on opposite sides of the second light receiver 323 in the Y direction. The shielding walls are provided to prevent light from entering the light receivers 312 and 323 directly from the light radiators 311, 321, and 322.

### Light transmissive member 400

The light transmissive member 400 transmits the measurement light radiated from the first measurement portion 310, the second light radiator 321, and the second light receiver 323, and the reflected light based on the measurement light. In the present embodiment, the light transmissive member 400 is made of polycarbonate. Note that the light transmissive member 400 is not necessarily made of polycarbonate, and may be made of any material capable of transmitting the measurement light and the reflected light described above. The light transmissive member 400 may be transparent or colored in black or the like as long as the light transmissive member 400 transmits the measurement light and the reflected light described above. The light transmissive member 400 has a disk-like exterior shape that is circular in the plan view viewed in the Z-axis direction, as shown in FIG. 4. A surface of the light transmissive member 400 that is the surface facing the positive end in the Z direction is the contact surface S3, which comes into contact with the object under inspection B1 in the measurement state, as shown in FIGS. 1, 3, and 4. The light transmissive member 400 includes a base portion 401, the four protrusions 402, and the one recess 403.

The base portion 401 has a convex-lens-like exterior shape protruding in the +Z direction as extending toward the center position in the plan view. In other words, the contact surface S3 has a round shape. The four protrusions 402 are provided at a central portion of the base portion 401. The thickness of the base portion 401 is about 0.4 mm (millimeters) at the central portion. The protrusions 402 have the same exterior shape. The protrusions 402 each have a circular shape viewed in the plan view and having a diameter smaller than that of the base portion 401. The distances from the center positions of the protrusions 402 to the center position of the base portion 401 are equal to each other. Two protrusions 402 are arranged in the Y-axis direction. The other two protrusions 402 are arranged in the X-axis direction. The four protrusions 402 are therefore arranged so as to be shifted from each other by 90° around the center of the base portion 401, as shown in FIGS. 1 and 4. A surface of each of the protrusions 402 that is the surface facing the positive end in the X direction has a round shape. In the present embodiment, the four protrusions 402 are not in contact with each other. The ratio of the radius of curvature of the contact surface S3 of the base portion 401 to the radius of curvature of a surface of each of the protrusions 402 that is the surface facing the positive end in the X direction is about 10:1. Note that the ratio is not limited to 10:1, and may be any other ratio that allows the radius of curvature of a surface of each of the protrusions 402 that is the surface facing the positive end in the X direction to be greater than the radius of curvature of the contact surface S3. The protrusions 402 each also act as a convex lens because of the shape of the protrusion 402. The protrusions 402 therefore serve to collect the reflected light toward the first light receiver 312 and the second light receiver 323 to improve the signal intensity.

The recess 403 is the section surrounded by the four protrusions 402. The "section surrounded by the four protrusions 402" has a broad meaning including a section having an outer edge a part of which is not in contact with the protrusions 402. For example, the recess 403 in the present embodiment has portions that are not surrounded by the protrusions 402 because any two of the protrusions 402 adjacent to each other are not in contact with each other. It can, however, be said that portions of the recess 403 which are the portions radially outward from the center of the recess 403, that is, the center of the base portion 401 and where the protrusions 402 are present are surrounded by the protrusions 402. The recess 403 therefore corresponds to the section surrounded by the four protrusions 402. The recess 403 does not protrude in the +X direction but is configured to be substantially parallel to the X-Y plane, as shown in FIG. 5.

The ratio between the radius of curvature of the contact surface S3 of the base portion 401 and the radius of curvature of a surface of each of the protrusions 402 that is the surface facing the positive end in the X direction is approximately 10:1, as described above. Arranging the four protrusions 402 side by side so as to form the one recess 403 at the center as described above allows the four protrusions 402 to apply a pressure having an appropriate magnitude to the object under inspection B1 in the measurement state. In the living body information measuring apparatus 10, in which the first measurement portion 310 and the second measurement portion 320 are arranged in the Y-axis direction, the light transmissive member 400 has a large diameter to realize radiation of the measurement light and reception of the reflected light in the two measurement portions 310 and 320. In other words, the radius of curvature of the base portion 401 is greater than that in a configuration in which only one measurement portion is provided. However, since the protrusions 402 each having a smaller radius of curvature are provided, application of an excessive load to the living body information measuring apparatus 10 to apply an appropriate pressure can be suppressed. Deformation of the light transmissive member 400 is therefore suppressed.

### Others

The battery 500 includes what is called a coin battery and a circuit that controls the output from the coin battery. The battery 500 supplies electric power to the first measurement portion 310, the second measurement portion 320, the analog front end IC 330, and the like.

The primary substrate 600 is provided with a circuit that controls the entire living body information measuring apparatus 10. For example, a circuit that counts the pulse rate and a circuit that calculates SpO₂ based on the output from the analog front end IC 330 are provided.

The waterproof cover 700 seals the portion where the first enclosure portion 101 and the second enclosure portion 102 are fitted to each other. The waterproof cover 700 is made, for example, of silicon.

### A3. Support of light transmissive member 400 performed by support portion 150:

The first opening 131 is disposed at a position corresponding to the first measurement portion 310 in the Z-axis direction in the sensor substrate 300, as shown in FIG. 8. The second opening 132 is disposed at a position corresponding to the second measurement portion 320 in the Z-axis direction in the sensor substrate 300. The beam portion 120 is configured as a section between the first opening 131 and the second opening 132, as described above. Therefore, when viewed in the direction in which the contact surface S3 and the object under inspection B1 face each other (hereinafter referred to as "facing direction"), the beam portion 120 is located between the first measurement portion 310 and the second measurement portion 320. Arranging the beam portion 120 as described above can prevent the beam portion 120 from blocking the radiation of the measurement light and the reception of the reflected light in the first measurement portion 310 and the second measurement portion 320. Furthermore, the presence of the beam portion 120 can prevent the light to be measured by the first measurement portion 310 and the light to be measured by the second measurement portion 320 from being mixed with each other and affecting the measurement result. Note that the facing direction corresponds to a direction substantially parallel to the Z-axis direction.

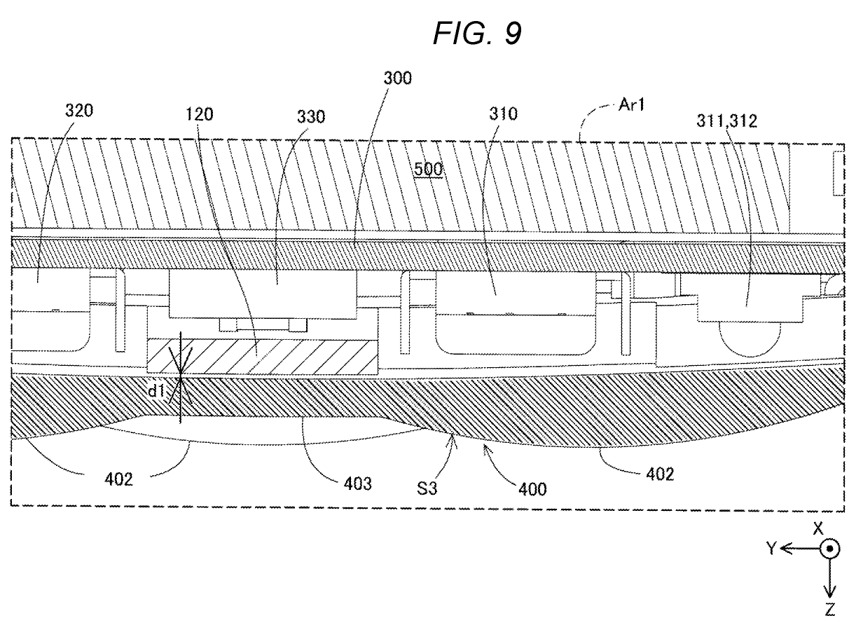

FIG. 9 is a partial cross-sectional view showing a detailed configuration of the living body information measuring apparatus 10. FIG. 9 is an enlarged view showing a region Ar1 indicated by the broken line in FIG. 5. Note that FIGS. 5 and 9 show the living body information measuring apparatus 10 before covered with the dressing member 20.

Before covered with the dressing member 20, a gap d1 is provided between the beam portion 120 and the sensor substrate 300, as shown in FIG. 9. In the present embodiment, the gap d1 is provided by forming an end surface of the light transmissive member 400 that is the surface facing the negative end in the Z direction in such a way that the end surface is not a planar surface but is a concave surface slightly shifted in the +Z direction as extending toward the center of the end surface. As described above, in the state in which the gap d1 is present, the beam portion 120 (support portion 150) does not come into contact with the light transmissive member 400 and therefore does not support the light transmissive member 400. The size of the gap d1 is, for example, 0.2 mm.

In the measurement state, however, when the living body information measuring apparatus 10 is pressed against the object under inspection B1 by the adhesive force produced by the dressing member 20 and a force (reaction force) greater than or equal to the predetermined threshold is applied by the object under inspection B1, the light transmissive member 400 bends in the −Z direction. In this case, the gap d1 disappears, and the light transmissive member 400 comes into contact with the beam portion 120. When the beam portion 120, which is supported by the base portion 110, comes into contact with the light transmissive member 400, the beam portion 120 supports the light transmissive member 400 and suppresses deformation of the light transmissive member 400 in the −Z direction. A situation in which the distance from the first measurement portion 310 and the second measurement portion 320 to the object under inspection B1 becomes smaller than an intended distance can therefore be avoided, so that a decrease in the measurement accuracy due to an excessive increase in the signal intensity can be suppressed. Furthermore, since the deformation of the light transmissive member 400 can be suppressed by the support portion 150, the light transmissive member 400 can be thin, and the distance from the first measurement portion 310 and the second measurement portion 320 to the object under inspection B1 can be shortened. The first measurement portion 310 and the second measurement portion 320 (first light receiver 312 and second light receiver 323) can therefore receive stronger signals.

In a state in which the light transmissive member 400 is in contact with the support portion 150 (beam portion 120), the distance from the contact surface S3 to the surfaces of the first measurement portion 310 and the second measurement portion 320 does not change even when a force (reaction force) that falls within a predetermined force range is applied to the light transmissive member 400 in the thickness direction of the light transmissive member 400. In the present embodiment, the force range described above is greater than or equal to 150 gf (gram weight) but smaller than or equal to 450 gf. In general, applying a pressure equal to the blood pressure to press the living body information measuring apparatus 10 against the object under inspection B1 allows measurement of a change accompanying a change in the volume of a blood vessel with noise suppressed. The "noise" corresponds, for example, to a change in the signal intensity resulting from the body motion. Assuming that the average blood pressure is 35 mmHg and the total area of the light transmissive member 400 and the enclosure surface S1 is 324 mm², the force described above is calculated to be 150 gf. Assuming that twice the value described above (300 gf) is a center value and a force of at least 150 gf is applied, the force range "greater than or equal to 150 gf but smaller than or equal to 450 gf" described above is derived.

For example, the type of the material of the first enclosure portion 101, the thickness or width of the beam portion 120, or other factors may be so adjusted that the distance from the contact surface S3 to the surfaces of the first measurement portion 310 and the second measurement portion 320 does not change even when a force (reaction force) within the predetermined force range is applied to the light transmissive member 400 in the thickness direction of the light transmissive member 400 in the state in which the light transmissive member 400 is in contact with the support portion 150 (beam portion 120) as described above.

### A4. Roles of protrusions 402 and recess 403:

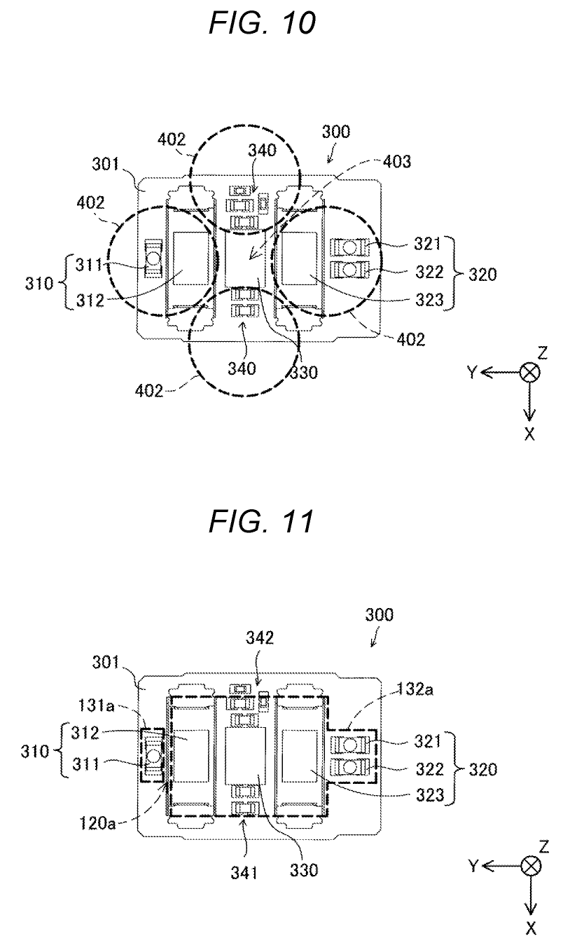

FIG. 10 illustrates the positional relationship of the four protrusions 402 and the recess 403 with the first measurement portion 310 and the second measurement portion 320. In FIG. 10, the four protrusions 402 and the recess 403 are diagrammatically drawn with broken lines, and are shown so as to overlap with the sensor substrate 300.

As described above, the four protrusions 402 and the recess 403 play a role of applying a pressure having an appropriate magnitude to the object under inspection B1 in the measurement state. In addition to the role described above, the four protrusions 402 and the recess 403 play the following roles (provide the following advantages).

In the first embodiment, the first measurement portion 310 is disposed at a position where the first measurement portion 310 overlaps with one of the protrusions 402 when viewed in the facing direction, as shown in FIG. 10. Similarly, the second measurement portion 320 is disposed at a position where the second measurement portion 320 overlaps with a protrusion 402 different from the protrusion 402 that overlaps with the first measurement portion 310 when viewed in the facing direction. In the arrangement described above, since the light radiators 311, 321, and 322 are disposed at positions where the light radiators 311, 321, and 322 overlap with the corresponding protrusions 402 when viewed in the facing direction in the measurement portions 310 and 320, the protrusions 402 can function as lenses to bring the measurement light into focus at the radiation positions on the object under inspection. Furthermore, since both the light radiators and the light receivers are disposed at positions where the light radiators and the light receivers overlap with the corresponding protrusions 402 when viewed in the facing direction in the measurement portions 310 and 320, the protrusions 402 can function as lenses to bring the measurement light into focus at the radiation positions on the object under inspection B1, and bring the reflected light into focus at the light receivers 312 and 323 to improve the intensity of the optical signals received thereby.

As described above, the direction indicator 103 indicates that the living body information measuring apparatus 10 is so attached to the object under inspection B1 that the lengthwise direction of the direction indicator 103 is parallel to the widthwise direction of the object under inspection B1. The living body information measuring apparatus 10 is therefore so attached to the object under inspection B1 that the Y-axis direction, which is one (first direction) of the directions in which pairs of protrusions 402 that sandwich the recess 403 are arranged, is parallel to the widthwise direction of the object under inspection B1, as shown in FIG. 2. In other words, it can be said that the direction indicator 103 indicates the mounting direction of the living body information measuring apparatus 10 in such a way that the first direction is parallel to the widthwise direction of the object under inspection. The thus configured direction indicator 103 allows an increase in the possibility of bringing the living body information measuring apparatus 10 into close contact with the object under inspection B1 in such a way that two protrusions 402 are arranged in parallel to the widthwise direction of the object under inspection B1. Therefore, even when the object under inspection B1 moves in the widthwise direction thereof, for example, even when the arm is twisted, a change in the posture of the living body information measuring apparatus 10 relative to the object under inspection B1 can be suppressed. The influence of the noise resulting from the body motion can therefore be further suppressed.

The living body information measuring apparatus 10 according to the first embodiment described above includes the support portion 150, which is disposed between the light transmissive member 400 and the enclosure 100 (first enclosure portion 101), and comes into contact with the light transmissive member 400 and supports the light transmissive member 400 when a force greater than or equal to the predetermined threshold is applied by the object under inspection B1 to the light transmissive member 400 in the measurement state, so that deformation such as a bend of the light transmissive member 400 can be suppressed when the object under inspection B1 is pressed with a pressing force greater than an intended force. A decrease in measurement accuracy due to the situation in which the distance from the first measurement portion 310 and the second measurement portion 320 to the object under inspection B1 becomes shorter than the intended distance and the signal intensity excessively increases accordingly can therefore be suppressed. Since the deformation of the light transmissive member 400 can be suppressed by the support portion 150, the light transmissive member 400 can be thin, and the distance from the first measurement portion 310 and the second measurement portion 320 to the object under inspection B1 can be shortened. The first measurement portion 310 and the second measurement portion 320 (first light receiver 312 and second light receiver 323) can therefore receive stronger signals.

In addition, since the support portion 150 includes the beam portion 120 located between the first measurement portion 310 and the second measurement portion 320 when viewed in the facing direction, and the base portion 110, which supports the beam portion 120, the situation in which the beam portion 120 blocks the radiation of the measurement light and the reception of the reflected light in the first measurement portion 310 and the second measurement portion 320 can be avoided. Furthermore, the presence of the beam portion 120 can prevent the light to be measured by the first measurement portion 310 and the light to be measured by the second measurement portion 320 from being mixed with each other and affecting the measurement result.

In addition, in the state in which the light transmissive member 400 and the support portion 150 (beam portion 120) are in contact with each other, the distance from the contact surface S3 to the surfaces of the first measurement portion 310 and the second measurement portion 320 does not change even when a pressing force within the force range greater than or equal to 150 gf but smaller than or equal to 450 gf is applied to the light transmissive member 400 in the thickness direction of the light transmissive member 400. Therefore, when a pressing force necessary for accurately measuring living body information is applied in the thickness direction of the light transmissive member 400, a change in the distance from the contact surface S3 to the surfaces of the first measurement portion 310 and the second measurement portion 320 can be suppressed, so that a decrease in the measurement accuracy of the living body information can be suppressed.

In addition, since the light transmissive member 400 has the four protrusions 402 and the recess 403, at least a part of which is surrounded by the four protrusions 402, at the contact surface S3, the four protrusions 402 can press the object under inspection B1 with a greater force than in a configuration in which the protrusions 402 are not provided. The influence of the noise resulting from the body motion or the like can thus be suppressed, and a signal resulting from living body information to be originally measured can be more accurately measured.

In addition, since at least the light radiators 311, 321, and 322 out of the light radiators 311, 321, and 322 and the light receivers 312 and 323 provided in the measurement portions 310 and 320 are disposed at positions where the light radiators 311, 321, and 322 each overlap with one of the four protrusions 402 when viewed in the facing direction, the corresponding protrusions 402 can function as lenses to bring the measurement light into focus at the radiation positions on the object under inspection B1.

In addition, since both the light radiators 311, 321, and 322 and the light receivers 312 and 323 provided in the measurement portions 310 and 320 are disposed at positions where the light radiators 311, 321, and 322 and the light receivers 312 and 323 each overlap with one of the four protrusions 402 when viewed in the facing direction, the corresponding protrusions 402 can function as lenses to bring the measurement light into focus at the radiation positions on the object under inspection B1, and the reflected light can be brought into focus at the light receivers 312 and 323 to improve the intensity of the optical signals received thereby.

In addition, since at least two protrusions 402 out of the four protrusions 402 are arranged side by side in the first direction (Y-axis direction), and the enclosure 100 (first enclosure portion 101) includes the direction indicator 103, which indicates the mounting direction of the living body information measuring apparatus 10 in such a way that the first direction is parallel to the widthwise direction of the object under inspection B1, the possibility of bringing the living body information measuring apparatus 10 into close contact with the object under inspection B1 in such a way that the two protrusions 402 are arranged in parallel to the widthwise direction of the object under inspection B1 can be increased. Therefore, even when the object under inspection B1 moves in the widthwise direction thereof, for example, even when the arm is twisted, a change in the posture of the living body information measuring apparatus 10 relative to the object under inspection B1 can be suppressed. The influence of the noise resulting from the body motion can therefore be further suppressed.

Furthermore, since the four protrusions 402 include a first pair of protrusions 402, which sandwich the recess 403 in a second direction, and a second pair of protrusions 402, which sandwich the recess 403 in a third direction perpendicular to the second direction, the protrusions 402 can be disposed in four directions shifted by 90° around the recess 403. Therefore, even when a body motion occurs in various directions, the influence of noise resulting from the body motion can be suppressed.

### B. Second embodiment:

FIG. 11 illustrates the positional relationship between a beam portion 120a in a second embodiment and each of the measurement portions 310 and 320. FIG. 11 shows two openings 131a and 132a and the beam portion 120a of a support portion in the second embodiment, which are drawn with broken lines and superimposed on a plan view of the sensor substrate 300 viewed in the +Z direction, as in FIG. 8.

The living body information measuring apparatus 10 according to the second embodiment differs from the living body information measuring apparatus 10 according to the first embodiment only in the configuration of the support portion 150. In the living body information measuring apparatus 10 according to the second embodiment, the same elements as those of the living body information measuring apparatus 10 according to the first embodiment have the same reference characters, and will not be described in detail. The support portion in the second embodiment differs from the support portion 150 in the first embodiment only in that the two openings 131 and 132 are replaced with the two openings 131a and 132a and the beam portion 120 is replaced with the beam portion 120a.

The opening 131a is located and sized so as to overlap with the first light radiator 311 but not to overlap with the first light receiver 312 when viewed in the facing direction, as shown in FIG. 11. The opening 132a is located and sized so as to overlap with the first light receiver 312 of the first measurement portion 310 and the entire second measurement portion 320 when viewed in the facing direction. Therefore, in the second embodiment, the beam portion 120a is formed so as to extend in the X-axis direction between the first light radiator 311 and the first light receiver 312 in the Y-axis direction.

The living body information measuring apparatus 10 according to the second embodiment described above provides the same advantages as those provided by the living body information measuring apparatus 10 according to the first embodiment.

### C. Other embodiments:

(C1) In each of the embodiments, in a state in which the light transmissive member 400 is in contact with the support portion 150 (beam portion 120), the distance from the contact surface S3 to the surfaces of the first measurement portion 310 and the second measurement portion 320 does not change even when a force (reaction force) that falls within a predetermined force range is applied to the light transmissive member 400 in the thickness direction of the light transmissive member 400, but not necessarily in the present disclosure. When a force (reaction force) within the force range described above is applied to the light transmissive member 400 in the thickness direction, the distance from the contact surface S3 to the surfaces of the first measurement portion 310 and the second measurement portion 320 may change. The configuration described above, in which the light transmissive member 400 is still supported by the support portion 150, also allows suppression of an excessive change in the distance from the contact surface S3 to the surfaces of the first measurement portion 310 and the second measurement portion 320, as compared with a configuration without the support portion 150.

(C2) In each of the embodiments, the support portion 150 includes the base portion 110 and the beam portion 120 or 120a, but not necessarily in the present disclosure. For example, even in a configuration in which the beam portion 120 or 120a is partially missing in the longitudinal direction and therefore divided into two sections, the base portion 110 supports each of the two divided sections in the form of a cantilever, so that the light transmissive member 400 can be supported. That is, in general, a support portion having any configuration in which the support portion comes into contact with the light transmissive member 400 when a force greater than or equal to a predetermined threshold is applied by the object under inspection B1 to the light transmissive member 400 in the measurement state to be capable of supporting the light transmissive member 400 may be used as the support portion in the present disclosure.

(C3) In the first embodiment, the beam portion 120 is disposed between the first measurement portion 310 and the second measurement portion 320 when viewed in the facing direction. In the second embodiment, the beam portion 120a is disposed between the first light radiator 311 and the first light receiver 312 when viewed in the facing direction. The present disclosure is not necessarily configured as described above. For example, in FIG. 10, the beam portion may be disposed at a position where the beam portion overlaps with a line that couples the gaps that are located between protrusions 402 adjacent to each other and located on a diagonal line when viewed in the facing direction.

The present disclosure is not limited to the embodiments described above, and can be implemented in various aspects to the extent that the various aspects do not depart from the intent of the present disclosure. For example, the present disclosure can also be implemented in the following aspects. To solve some or all of the problems described in the present disclosure, or to achieve some or all of the advantages of the present disclosure, the technical features of the embodiments described above that correspond to the technical features in each of the following aspects can be replaced or combined with each other as appropriate. In addition, the technical features can be deleted as appropriate unless described as essential features in the present specification.

### D. Other aspects:

(1) According to an aspect of the present disclosure, there is provided a living body information measuring apparatus used in close contact with an object under inspection with the aid of a dressing member. The living body information measuring apparatus includes: an enclosure having an enclosure surface that comes into contact with the object under inspection in a measurement state in which living body information on the object under inspection is measured; a light measurement portion disposed in the enclosure and configured to radiate measurement light to the object under inspection and receive reflected light; a light transmissive member configured to transmit the measurement light and the reflected light, and including multiple protrusions configured to press the object under inspection in the measurement state, the multiple protrusions located at the contact surface; and a support portion disposed between the light transmissive member and the enclosure, and configured to come into contact with the light transmissive member to support the light transmissive member when a force greater than or equal to a predetermined threshold is applied by the object under inspection to the light transmissive member in the measurement state.

The living body information measuring apparatus according to the aspect includes the support portion, which is disposed between the light transmissive member and the enclosure, and comes into contact with the light transmissive member to support the light transmissive member when a force greater than or equal to the predetermined threshold is applied by the object under inspection to the light transmissive member in the measurement state, so that deformation such as a bend of the light transmissive member can be suppressed when the object under inspection is pressed with a pressing force greater than an intended force. A decrease in measurement accuracy due to the situation in which the distance between the object under inspection and the light measurement portion becomes shorter than an intended distance and the signal intensity excessively increases accordingly can therefore be suppressed. Furthermore, since the deformation of the light transmissive member can be suppressed by the support portion, the light transmissive member can be thin, and the distance between the object under inspection and the detection portion can be shortened. The light measurement portion can therefore receive a stronger signal.

(2) In the living body information measuring apparatus according to the aspect described above, the light measurement portion includes a first light radiator configured to radiate the measurement light, and a first light receiver configured to receive the reflected light, and the support portion includes a beam portion located between the first light radiator and the first light receiver when viewed in a facing direction in which the contact surface and the object under inspection face each other, and a base portion configured to support the beam portion.

The living body information measuring apparatus according to the aspect, in which the support portion includes the beam portion located between the first light radiator and the first light receiver when viewed in the facing direction, and the base portion configured to support the beam portion, can avoid a situation in which the support portion prevents the first light radiator from radiating the measurement light and the first light receiver from receiving the reflected light. In addition, the presence of the beam portion can prevent the measurement light radiated from the first light radiator from directly entering the first light receiver.

(3) In the living body information measuring apparatus according to the aspect described above, the light measurement portion includes a first measurement portion including a first light radiator configured to radiate the measurement light and a first light receiver configured to receive the reflected light, and a second measurement portion including a second light radiator configured to radiate the measurement light and a second light receiver configured to receive the reflected light, and the support portion includes a beam portion located between the first measurement portion and the second measurement portion when viewed in a facing direction in which the contact surface and the object under inspection face each other, and a base portion configured to support the beam portion.

The living body information measuring apparatus according to the aspect, in which the support portion includes the beam portion located between the first measurement portion and the second measurement portion when viewed in the facing direction, and the base portion configured to support the beam portion, can avoid the situation in which the beam portion blocks the radiation of the measurement light and the reception of the reflected light in the first and second measurement portions. Furthermore, the presence of the beam portion can prevent the light to be measured by the first measurement portion and the light to be measured by the second measurement portion from being mixed with each other and affecting the measurement result.

(4) In the living body information measuring apparatus according to the aspect described above, in a state in which the light transmissive member and the support portion are in contact with each other, a distance between the contact surface and a surface of the light measurement portion does not change even when a force within a predetermined force range is applied to the light transmissive member in a thickness direction of the light transmissive member, and the force range is greater than or equal to 150 gf but smaller than or equal to 450 gf.

In the living body information measuring apparatus according to the aspect, in the state in which the light transmissive member and the support portion are in contact with each other, the distance between the contact surface and the surface of the light measurement portion does not change even when a pressing force within the force range greater than or equal to 150 gf but smaller than or equal to 450 gf is applied to the light transmissive member in the thickness direction of the light transmissive member. Therefore, when a pressing force necessary for accurately measuring living body information is applied in the thickness direction of the light transmissive member, a change in the distance between the contact surface and the surface of the light measurement portion can be suppressed, so that a decrease in the measurement accuracy of the living body information can be suppressed.

In addition to the aspects described above, the present disclosure can be implemented in an aspect of a method for manufacturing a living body information measuring apparatus, a light transmissive member used in a living body information measuring apparatus, and the like.

What is claimed is:

1. A living body information measuring apparatus comprising:
an enclosure having an enclosure surface that comes into contact with an object under inspection in a measurement state in which living body information on the object under inspection is measured;
a light measurement portion disposed in the enclosure and configured to radiate measurement light to the object under inspection and receive reflected light;
a light transmissive member configured to transmit the measurement light and the reflected light, and including multiple protrusions configured to press the object under inspection in the measurement state, the multiple protrusions located at the contact surface; and
a support portion disposed between the light transmissive member and the enclosure, and configured to come into contact with the light transmissive member to support the light transmissive member when a force greater than or equal to a predetermined threshold is applied by the object under inspection to the light transmissive member in the measurement state.

2. The living body information measuring apparatus according to claim 1, wherein
the light measurement portion includes a first light radiator configured to radiate the measurement light, and a first light receiver configured to receive the reflected light, and
the support portion includes
a beam portion located between the first light radiator and the first light receiver when viewed in a facing direction in which the contact surface and the object under inspection face each other, and
a base portion configured to support the beam portion.

3. The living body information measuring apparatus according to claim 1, wherein
the light measurement portion includes
a first measurement portion including a first light radiator configured to radiate the measurement light and a first light receiver configured to receive the reflected light, and
a second measurement portion including a second light radiator configured to radiate the measurement light and a second light receiver configured to receive the reflected light, and
the support portion includes
a beam portion located between the first measurement portion and the second measurement portion when viewed in a facing direction in which the contact surface and the object under inspection face each other, and
a base portion configured to support the beam portion.

4. The living body information measuring apparatus according to claim 1, wherein
in a state in which the light transmissive member and the support portion are in contact with each other, a distance between the contact surface and a surface of the light measurement portion does not change even when a force within a predetermined force range is applied to the light transmissive member in a thickness direction of the light transmissive member, and
the force range is greater than or equal to 150 gf but smaller than or equal to 450 gf.
